(19) United States
(12) Patent Application Publication
(10) Pub. No.: US 2011/0178480 A1
(43) Pub. Date: Jul. 21, 2011

(54) Shunt Apparatus For Treating Obesity By Extracting Food

(75) Inventors: Kenneth Solovay, Weston, FL (US); Samuel Klein, Clayton, MO (US); Stephen B. Solomon, New York, NY (US); Moshe Shike, Larchmont, NY (US)

(73) Assignee: ASPIRE BARIATRICS, LLC, Philadelphia, PA (US)

(21) Appl. No.: 13/073,720

(22) Filed: Mar. 28, 2011

Related U.S. Application Data
(62) Division of application No. 11/824,953, filed on Jul. 3, 2007.
(60) Provisional application No. 60/806,556, filed on Jul. 5, 2006.

(51) Int. Cl. A61M 1/00

(52) U.S. Cl. 604/317

(57) ABSTRACT

To treat obesity, a tube is positioned so that it passes through a patient's abdominal wall into the upper digestive system of the patient. The patient is allowed to carry out his/her everyday affairs including ingesting food. After ingestion, food is extracted by pumping it out of the upper digestive system through the tube. The embodiments described herein take advantage of some properties of ePTFE to provide a number of significant advantages.

### RELATED APPLICATIONS

This application claims the benefit of and priority to U.S. Provisional Application No. 60/806,556 filed on Jul. 5, 2006, the entirety of which is incorporated by reference herein.

### BACKGROUND OF THE INVENTION

Obesity is a major health problem in the United States and other countries. The National Health and Nutrition Examination Survey (1988-1994) reported that approximately 20-25% of Americans are obese, while another study estimated the percentage of overweight Americans to be between 60% and 65% (Flegal K M, Carroll M D, Ogden C L, Johnson C L “Prevalence and trends in obesity among US adults, 1999-2000” JAMA 2002; 288:1723-1727). Obesity can cause numerous health problems, including diabetes, degenerative joint disease, hypertension, and heart disease. Weight reduction can be achieved by increased caloric expenditure through exercise and/or by reduced caloric consumption through diet. However, in most cases, weight gain often recurs and improvements in related co-morbidities are often not sustained.

Surgical procedures present an increasingly common solution for obese patients. Surgical procedures include, for example, stapled gastroplasty, banded gastroplasty, gastric banding, gastric bypass surgery, and bilopancreatic bypass. However, these surgical procedures are invasive, risky and expensive to perform, and many patients regain a substantial portion of the lost weight.

### SUMMARY OF THE INVENTION

The present invention is directed to apparatuses and methods for treating obesity or facilitating weight loss. A passageway is introduced into a patient's upper digestive system such that it passes through the patient's abdominal wall. The patient is allowed to carry out his/her everyday affairs including ingesting food. After the patient has ingested food, the food is extracted by pumping it out of the upper digestive system through the passageway. This approach is less invasive than the procedures discussed above, easy to perform, easy to reverse and has successfully resulted in significant weight loss in obese patients.

### BRIEF DESCRIPTION OF THE DRAWINGS

FIG. 1 is a schematic view of a first embodiment of the present invention installed in a patient;

FIG. 1A is a schematic view of a tube;

FIG. 1B is an alternate view of a tube;

FIG. 1C is a cross sectional schematic view of a tube;

FIG. 2 is a schematic view of a variation of an embodiment of the present invention that uses a manual bulb pump;

FIG. 3 is a schematic view of a variation of an embodiment of the present invention that uses a syringe as a pump;

FIG. 4 is a schematic view of a variation of an embodiment of the present invention that uses a bag connected to a pump;

FIG. 5 is a schematic view of how an embodiment of the present invention can be cleaned;

FIG. 6 is a schematic view of a second embodiment of the present invention that uses an inflated balloon anchor;

FIG. 7 is an axial cross sectional schematic view showing valves provided in the lumens of a tube in an embodiment of the present invention;

FIG. 8 is a schematic view of a third embodiment of the present invention having a tube with two balloons attached to that portion of the tube that is disposed within the patient's digestive system;

FIG. 9 is a schematic view of a fourth embodiment of the present invention having a tube with a curved configuration and a plurality of holes in a sidewall;

FIG. 10 is a schematic view of a fifth embodiment of the present invention having a tube with a curved configuration, multiple holes in a sidewall, and a morcellation device housed within a cage at its distal end portion;

FIG. 11 is a schematic view of the proximal end portion of a tube lying substantially flush with a patient's abdominal wall;

FIG. 12 is a schematic view of a luer lock at the proximal end portion of a tube;

FIG. 13 is a schematic view of a variation of an embodiment of the present invention having a tube with a funnel shaped tip;

FIG. 14 is a schematic view of a sixth embodiment of the present invention having two intake tubes;

FIG. 15A and FIG. 15B are schematic views of an embodiment of the present invention installed in a patient illustrating how the apparatus accommodates changes in thickness of the abdominal wall of a patient;

FIG. 16 illustrates how an embodiment of the present invention installed in a patient is used.

FIGS. 17A and 17B are isometric and plan views, respectively, of another embodiment of a gastrostomy tube, which has a helical external support structure.

FIG. 18 depicts a mechanism for installing the gastrostomy tube of FIG. 17.

FIGS. 19A-E are exploded, partially assembled section, and fully assembled section views of low-profile termination for the gastrostomy tube of FIG. 17.

FIG. 20 depicts a low cost alternative embodiment of a gastrostomy tube.

### DETAILED DESCRIPTION OF PREFERRED EMBODIMENTS

As used herein, the term “food” includes both solid and liquid substances that have been ingested by the patient, the term “ingest” or “ingested” includes eating and drinking, and the term “upper digestive system” includes the stomach 3, duodenum 4 and proximal jejunum of the patient.

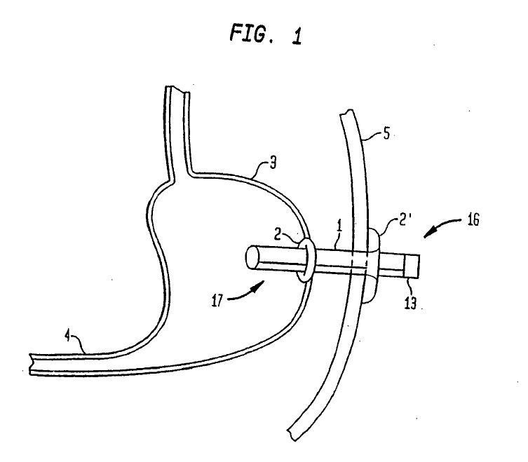

In a first embodiment of the present invention as shown in FIG. 1, a transabdominal tube 1 is placed through a patient's abdominal wall such that a distal end portion 17 of the tube 1 is disposed inside the stomach 3 of the patient and a proximal end portion 16 of the tube 1 extends out from the skin 5 of the patient. The tube 1 preferably has a diameter that is 20 to 36 French in size (1 French=⅓ mm). Most preferably, the tube has a relatively large inner diameter (e.g., greater than 6.3 mm internal diameter) and the tube resists collapsing when extraction is performed. Optionally, the tube 1 may be stiffened, made durable and less collapsible by, for example, braiding the tube using nylon. Alternatively, the tube may be wrapped with wire material. Suitable materials for the tube 1 include polyurethane, silicone and other similar materials. The tube 1 may be opaque.

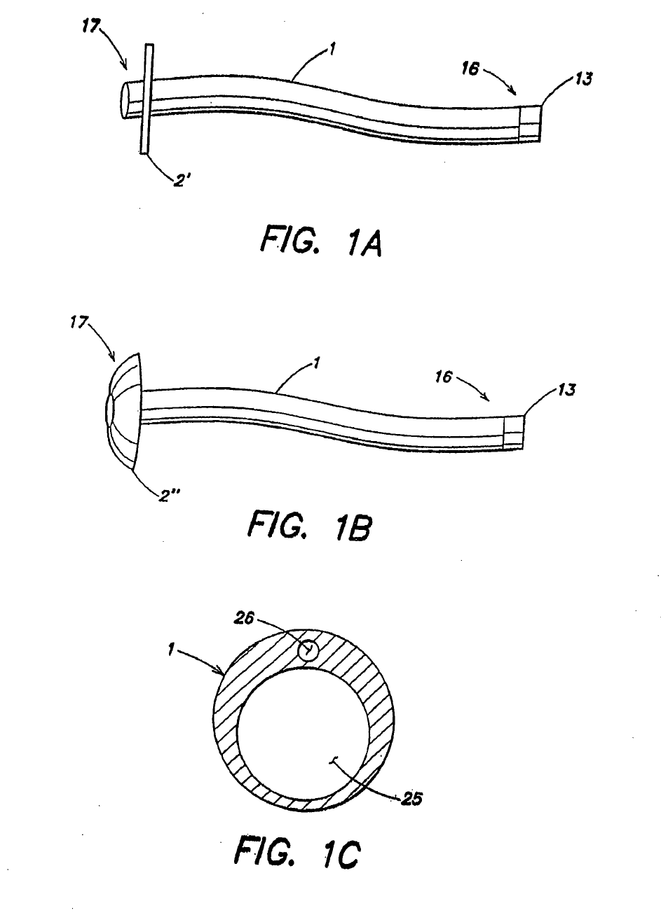

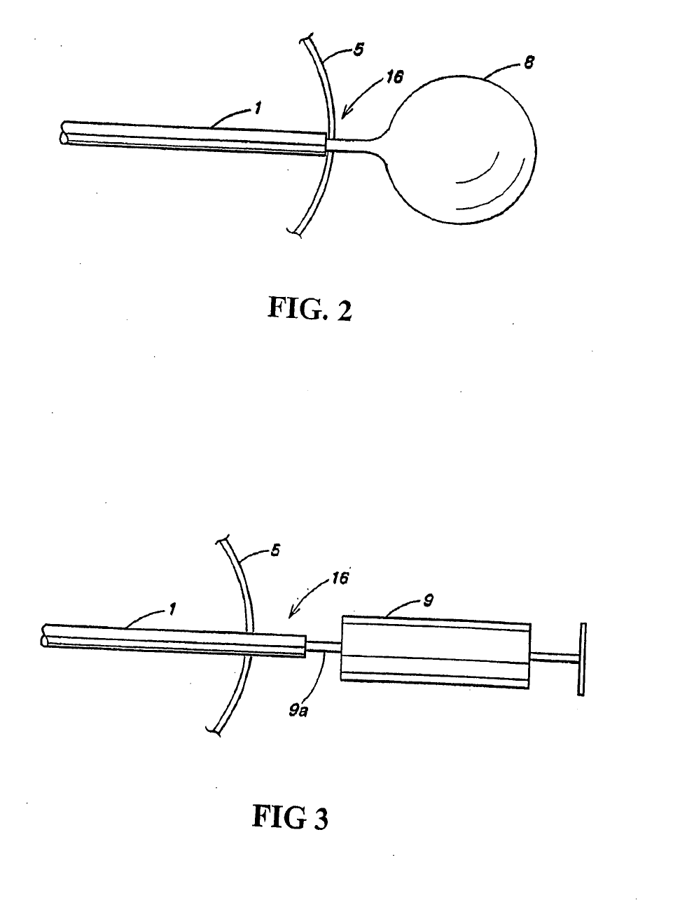

A retention member is attached to the tube 1 to prevent the tube 1 from falling out of the patient. In some embodiments, the retention member is inflatable such as the inflation portion 2 (balloon anchor) shown in FIG. 1. As shown in FIG. 1, the inflation portion 2 is provided at the distal end portion 17 of the tube 1 to prevent the tube 1 from coming out of the stomach 3. FIG. 1 also illustrates a non-inflatable retention member flange 2′ at the proximal end portion 16 of the tube 1 to prevent the tube 1 from falling into the patient's upper digestive system. The flange 2′ can prevent inadvertent dislodgement of the tube 1 into the interior of the patient's body. A cap 13 is detachably provided at the end of the proximal end portion 16 and seals the tube 1 when it is attached. The cap 13 is removed when a pump 8, 9 (shown in FIGS. 2 and 3, respectively) is attached to the tube 1 to remove food from the upper digestive system of the patient.

Reference is now made to methods which may be used to insert the tube 1. These methods entail less risk of complications and less cost than conventional, surgical methods of treating obesity, and patients who undergo these treatments are typically discharged the same day of the operation. These methods are therefore especially advantageous for use in treating obese patients because such patients are at increased risk for surgical complications due to their obesity.

The tube 1 may be inserted, for example, through a procedure similar to insertion of feeding tubes by Percutaneous Endoscopic Gastrostomy (PEG). A variety of methods of performing PEG are well known in the art, and any one of the methods may be used to insert the tube 1. PEG procedures have been successfully completed in over 90 percent of attempts. PEG may be performed under conscious sedation induced by, for example, meperidine and midazolam. According to one method of PEG known as the pull method, an endoscope is inserted into the stomach through the mouth of the patient. The stomach is insufflated by blowing air into the stomach through the endoscope. The insufflation brings the stomach in apposition to the abdominal wall and allows for direct access from the skin to the stomach of the patient.

An insertion site is located by surveying the interior of the stomach with the endoscope. The endoscope is then used to illuminate the selected insertion site in such a way that the light of the endoscope is visible from outside of the patient's body through the skin of the patient.

An incision is made at the place on the patient's skin indicated by the light from the endoscope and at the corresponding location on the exterior wall of the stomach. A cannula is then inserted through the incision and a guide wire is inserted into the stomach through the cannula. Graspers on the end of the endoscope grab hold of the distal portion of the guide wire in the stomach and the endoscope is withdrawn from the patient while the graspers hold the guide wire. The guide wire is of sufficient length to allow a proximal portion of it to extend out of the patient from the cannula after the distal portion is withdrawn from the stomach and through the patient's mouth by the endoscope.

The end of the guide wire extending out from the patient's mouth is attached to the proximal end of the tube 1, which is drawn though the mouth and esophagus and into the stomach of the patient by pulling on the proximal end of the guide wire. The tube 1 is then pulled through the incision in the stomach and skin of the patient until only the distal end portion 17 and the inflation portion 2 of the tube 1 remain inside of the stomach. Optionally, the tube 1 may have a coned tip to help move the tube 1 through the incision in the stomach. Optionally, a wire at the tip of the cone may be used for pulling the tube 1 through the incision. Once the tube 1 is in place, the coned tip may be cut off. The cannula is removed as the proximal end 16 of the tube 1 is drawn through the incision in the stomach, and is removed entirely when the proximal end 16 of the tube 1 is disposed at the patient's skin. The inflation portion 2 of the tube 1 is then inflated by introducing fluid into the inflation portion 2 through the inflation lumen 26. The inflated inflation portion holds the tube 1 in place and the guide wire is removed from the tube 1. A non-inflatable retention member such as a flange 2′ may be placed on the proximal end portion 16 of the tube 1 to keep the tube 1 disposed at the patient's skin.

An alternate method of PEG known as push PEG may also be used to insert the tube 1. The tube 1 is pushed through the incision in the stomach and the skin of the patient until it is disposed as described hereinabove with respect to the pull method.

A third method which may be used for inserting the tube 1 via PEG is known as the Russell method. As with both the push method and the pull method, the insertion site is located via endoscopy. An incision is made in the skin and stomach and a guide wire is inserted through the incision into the stomach via a cannula or needle. A dilator (or introducer) with a peel away sheath is guided along the guide wire and inserted into the stomach. After the dilator (introducer) and sheath are inside the gastric lumen, the dilator is removed and the tube 1 is inserted along the guide wire and through the peel away sheath. The sheath is then peeled away and the tube 1 is fixed in place.

The tube 1 may also be inserted without using an endoscope, for example, through a procedure similar to insertion of feeding tubes by Percutaneous Radiological Gastrostomy (PRG). According to PRG, the stomach is insufflated via a nasogastric tube. Organs which may be interposed between the stomach and the abdominal wall, such as the colon, are excluded by CT scan or ultrasonography. Exclusion of interposed organs may also be accomplished after insufflation by fluoroscopy. The selection of the insertion site is also determined by fluoroscopy or a similar method.

After the insertion site has been located, the tube 1 may be inserted transabdominally as in the Russell method of PEG. Alternatively, a guide wire may be inserted as in the endoscopic pull method. The wire is then maneuvered through the stomach and esophagus and out of the patient's mouth and is used to guide the tube 1 back through the mouth, esophagus and stomach and out of the insertion site (see, e.g., Mustafa N. Zmen et al. “Percutaneous Radiologic Gastrostomy” European Journal of Radiology 43:186-95).

The tube 1 may be inserted surgically. One suitable surgical technique that may be used to insert the tube 1 is the laparoscopic method. In this method, after pneumoperitoneum has been created, a 5 mm trocar is used to grasp a site on the anterior stomach wall that is appropriate for tube placement without excessive tension on the stomach. A skin incision down to the rectus sheath is made. A trocar is placed through the rectus sheath and the stomach wall is grasped and pulled upwards. An incision is made in the stomach and the tube 1 is inserted. Using the retention member at the distal end portion 17 of the tube 1, the stomach is brought snugly against the abdominal wall. The tissue is sutured around the tube 1. (See, e.g., Andrew Luck et al. “Laparoscopic Gastrostomy: Towards the Ideal Technique” Aust. N. Z. J. Surg. (1998) 68:281-283).

The tube 1 may be inserted in other portions of the upper digestive system besides the stomach. For example, direct jejunostomy, wherein a tube is inserted transabdominally into the jejunum, may be accomplished through methods similar to those described hereinabove with reference to gastrostomy tube placement. The retention member of the device should generally be smaller for jejunostomy procedures to avoid irritation of the jejunum or obstruction of the jejunal lumen.

FIG. 1 illustrates an inflatable retention member, i.e. the inflation portion 2, that is attached to the tube 1 to prevent the tube 1 from falling out of the patient. FIGS. 1, 1A and 1B illustrate two alternative non-inflatable retention members that may be used in place of and/or in addition to the inflatable portion 2. FIGS. 1 and 1A illustrate a flange 2′ and FIG. 1B illustrates a dome 2″. A flange 2′ or dome 2″ that is located at the distal end portion 17 of the tube 1 helps to prevent the tube 1 from coming out of the stomach 3 or other section of the upper digestive system. A flange 2′ or dome 2″ that is located at the proximal end portion 16 of the tube 1 helps to prevent the tube from falling into the patient's upper digestive system.

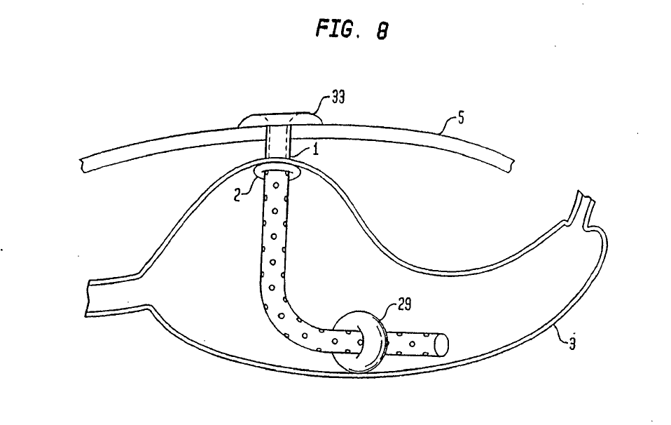

When an inflatable retention member is used, the tube 1 preferably has an inflation lumen 26 so that the inflatable retention member can be inflated. FIG. 1C shows a cross section of the tube 1 taken perpendicular to the axis of tube 1. Inflation lumen 26 extends from the inflation portion 2 to the proximal end portion 16 of the tube 1 and is a pathway for introducing fluid, such as water or air, to the inflation portion 2 from outside of the patient. Removal lumen 25 extends from the proximal end portion 16 to the distal end portion 17 of the tube 1 and is a pathway for the removal of food from the stomach 3 or other part of the upper digestive system of the patient. The inflation lumen 26 is preferably minimal in size to allow the removal lumen 25 to be as wide as possible within the tube 1. In the illustrated embodiment, valves 15, 27 are provided in lumens 25, 26, respectively, as shown in FIG. 7. With the non-inflatable retention members 2′ and 2″ shown in FIGS. 1A and 1B, the second lumen 26 in tube 1 can be eliminated.

Inflatable retention members are suitable for use with procedures similar to the push method, while either inflatable or rigid retention members are suitable for use with procedures similar to the pull method. One example of a tube that has an inflatable retention member is taught in Tiefenthal et al. (U.S. Pat. No. 6,506,179), the entire contents of which are incorporated herein by reference. An alternative deformable retention member is taught in Snow et al. (U.S. Pat. No. 6,077,250), the entire contents of which are incorporated herein by reference.

Retention members that may be deformed in situ allow the tube 1 to be removed without additional endoscopy. The retention member is deflated or deformed and the tube 1 is pulled out using traction. In cases where the retention member is rigid, the tube 1 may be cut close to the skin and removed endoscopically.

It is preferable for the stomach to be positioned up against the inner abdominal wall. This may be accomplished by insufflation during the tube placement procedure and after the tube 1 has been placed due to the retention member. For example, as shown in FIG. 1, retention members at the proximal end portion 16 and distal end portion 17 of the tube 1 anchor the stomach up against the abdominal wall. The stomach may also be anchored to the abdominal wall by gastropexy, which may prevent complications arising from tube placement and may facilitate the placement procedure. In addition, jejunopexy is important in jejunostomy procedures in order to secure the jejunum during the tube placement procedure (see Zmen et al., supra). For example, to secure the stomach or jejunum to the abdominal wall, T-shaped metal or nylon fixing members may be inserted trans-gastrically or trans-jejunally close to the tube insertion site. The fixing members assume a T shape after insertion and are tied near to the skin. Four fixing members are typically disposed in a square pattern around the tube insertion site to secure the stomach or jejunum. (See, e.g., F. J. Thornton et al. “Percutaneous Radiologic Gastrostomy with and without T-Fastener Gastropexy: a Randomized Comparison Study” Cardiovasc Intervent Radiol. 2002 November-December; 25(6):467-71).

Reference is now made to various forms of pumps which are attachable to the proximal end portion 16 of the tube 1. Any conventional pump, the construction of which will be readily understood to one skilled in the art, may be used. FIGS. 2 and 3, for example, display pumps 8 and 9 which are attachable to the proximal end portion 16 of the tube 1 for removal of food from the stomach 3 or upper digestive system of the patient. It would be suitable to use a pump that extracts more than 750 ml of food from the upper digestive system of a patient within 30 minutes or less. The pump may be operated intermittently to prevent tube collapse, tube clogging or mucosal irritation. The pump may be manual or battery operated. Optionally, a rechargeable power supply may be incorporated into the pump, and the pump may be configured to be carried on a patient's belt.

FIG. 2 depicts a manual bulb pump 8 that is attached to the proximal end portion 16 of the tube 1 and is operated to remove food from the patient's upper digestive system through the tube 1. The manual bulb pump 8 preferably comprises silicone rubber or a similar flexible material so as to permit the contents of the bulb pump 8 to be evacuated by squeezing the bulbous end of the bulb pump 8. The circumference of a tapered end essentially corresponds to an interior circumference of the lumen 25 of the tube 1. To operate the manual bulb pump 8, air is first evacuated from the bulb pump 8 by squeezing the bulb, and then the tapered end of the bulb pump 8 is inserted into the lumen 25 of the proximal end portion 16 of the tube 1 so as to create a seal between the tapered end and the tube 1. The bulb is then released to allow it to re-inflate. The negative pressure in the bulb pump 8 (when it is released) causes food to flow out from the upper digestive system toward the proximal end portion 16 of the tube 1 and into the bulb of the manual bulb pump 8. The bulb pump 8 is then disengaged from the tube 1 and the removed food is evacuated from the bulb. The cycle may be repeated until a desired amount of food is removed from the upper digestive system of the patient.

FIG. 3 depicts another pumping arrangement in which a pump in the form of a syringe 9 is attached to the proximal end portion 16 of the tube 1 and is operated to remove food from the patient's upper digestive system through the tube 1. The syringe 9 preferably comprises a tapered end portion with an aperture at the distal end thereof. The circumference of the tapered end portion 9a corresponds to the interior circumference of the lumen 25 of the tube 1. To operate the syringe 9 to remove food from the upper digestive system of the patient, the contents (air or food) of the syringe 9 are evacuated by depressing the plunger. The tapered end portion 9a of the syringe 9 is inserted into the proximal end portion 16 of the tube 1 so as to create a seal between the tapered end portion 9a and the tube 1. The plunger of the syringe 9 is then withdrawn so as to create negative pressure to draw food out from the upper digestive system through the tube 1 and into the syringe 9. The syringe 9 is then disengaged from the tube 1 and evacuated by, for example, depressing the plunger thereof. 60 cc is an example of a suitable size for the syringe 9. The cycle may be repeated until a desired amount of food is removed from the upper digestive system of the patient.

The manual bulb pump 8 and syringe 9 may be activated by the patient or by a health care provider at a predetermined time after eating. The predetermined time is preferably set by a physician and, for example, may be 20-30 minutes. A physician may also determine a maximum volume of food to be removed from the upper digestive system of the patient after each meal. The maximum volume may be set in terms of a maximum number of pumping cycles which is told to the patient or health care provider if the pump 8, 9 is manually operated.

In a preferred embodiment, the pump that is used to extract food from the patient's upper digestive system periodically reverses direction and pumps air and/or water into the upper digestive system of the patient during the periods of reverse operation. The air and/or water helps to solubilize or breakdown the food in the upper digestive system so that it can be pumped out easily. In addition, the air and/or water helps prevent the tube 1 from being suctioned up against the stomach wall while food is extracted from the upper digestive out through the tube 1. For example, every seven seconds of pumping may be followed by two seconds of reverse operation.

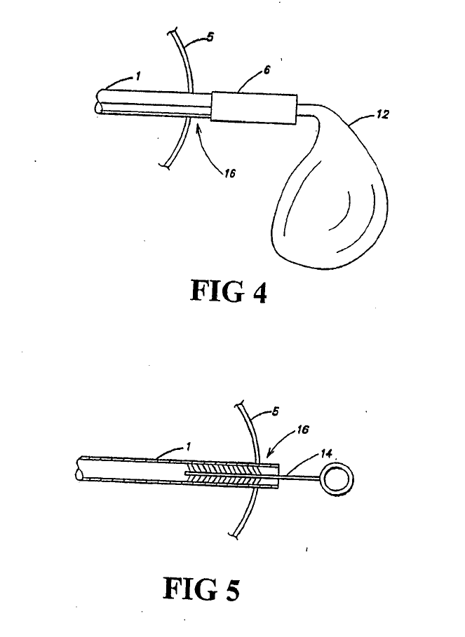

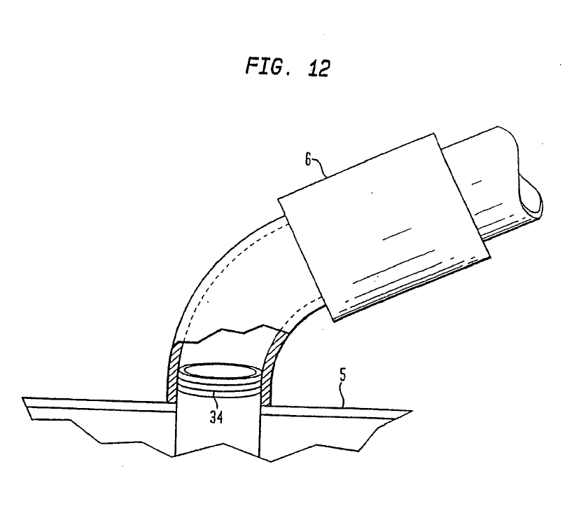

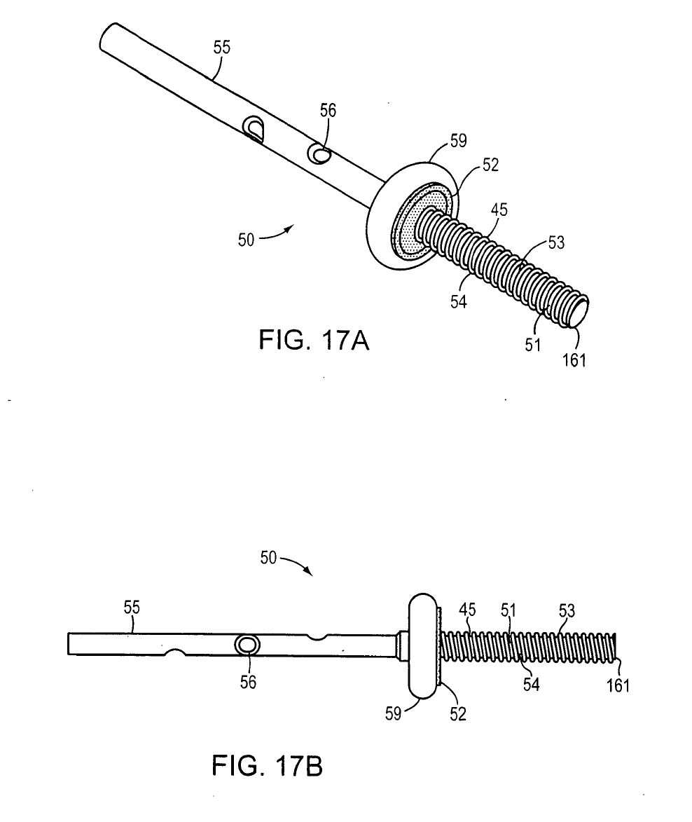

FIG. 4 illustrates a variation of an embodiment of the present invention in which the extracted food is evacuated from a pump 6 into a bag 12 that is attached to the pump 6. As shown in FIG. 4, after the food is pumped out of the upper digestive system of the patient by the pump 6, the food may be stored in a bag 12 that is attachable to the proximal end portion of the pump 6. The bag 12 may be opaque, scented, biodegradeable and worn by the patient on a belt or other strap. Alternatively, as shown in FIGS. 11 and 16, the food may be pumped from the patient's upper digestive system into the pump 6 and then into a tube 28 attached to the pump 6. The contents of the tube 28 attached to the pump 6 may be emptied into a toilet. The tube 28 may be opaque, scented, biodegradeable and flushable down the toilet.

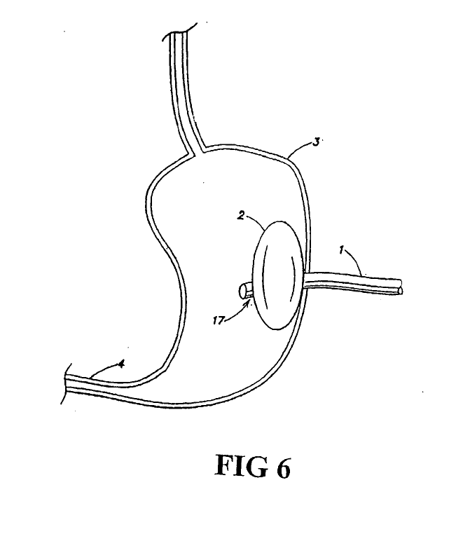

FIG. 5 illustrates a cleaning device being used to clean the tube 1 after food has been extracted from the patient's upper digestive system through the tube 1. As shown in FIG. 5, the tube 1 may be cleaned using a brush 14 that is adapted to clean the inside of the tube 1. The pump 6, manual bulb pump 8 and syringe 9 may be cleaned by flushing them with saline and/or a disinfectant solution after use.

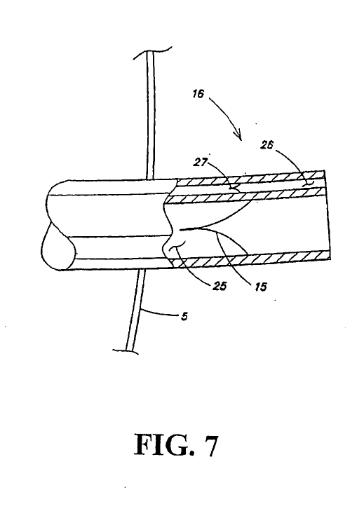

FIG. 6 illustrates a second embodiment of the present invention in which a feeling of satiety is created in the patient by inflating the balloon anchor. Creating a feeling of satiety curbs the patient's hunger and desire to eat food thereby allowing the patient to eat less and lose weight. As shown in FIG. 6, the inflation portion 2, which is the retention member that holds the tube 1 in the patient's stomach, also serves the function of decreasing stomach capacity to create a feeling of satiety when it is inflated. The inflation portion 2 may be variably inflated by adding or removing fluid through the inflation lumen 26 of the tube 1 (shown in FIG. 1C).

FIG. 7 shows an axial cross sectional view of the tube 1 extending out from the skin 5 of the patient in which the removal lumen 25 and the inflation lumen 26 are visible. In a feature which may be incorporated into any of the various embodiments of the present invention, a valve 15 is provided at the proximal end portion 16 of the tube 1 in the removal lumen 25. The valve 15 ordinarily prevents food from leaving the tube 1. The valve 15 is opened when a pump is attached to the proximal end portion 16 of the tube 1. For example, the tapered end portion of the manual bulb pump 8 (shown in FIG. 2) and the tapered end portion of the syringe 9 (shown in FIG. 3) each push open the valve 15 when they are inserted into the proximal end portion 16 of the tube 1. When the valve 15 is opened by the ends of the pumps, food can be removed as described hereinabove. A cap 13 (shown in FIG. 1) is preferably placed on the proximal end portion 16 of the tube 1 when a pump is not attached. The cap 13 may be pressed onto the end of the tube 1, threaded on the end of the tube 1, or may have projections which are frictionally inserted into the ends of lumens 25, 26 to seal them in a closed condition.

FIG. 7 also shows a valve 27 provided at the proximal end portion 16 of the tube 1 in the inflation lumen 26. The valve 27 prevents the fluid used to inflate the inflation portion 2 from escaping the inflation portion 2 through the inflation lumen 26. That is, the valve 27 prevents the inflation portion 2 from deflating. If it becomes necessary to deflate the inflation portion 2 to remove the tube 1 from the upper digestive system of the patient, or to further inflate the portion 2, a needle on a syringe may be inserted into the inflation portion 26 so as to open the valve 27 by pushing the needle through the valve members. The fluid used to inflate the inflation portion 2 may then be removed or added with the syringe.

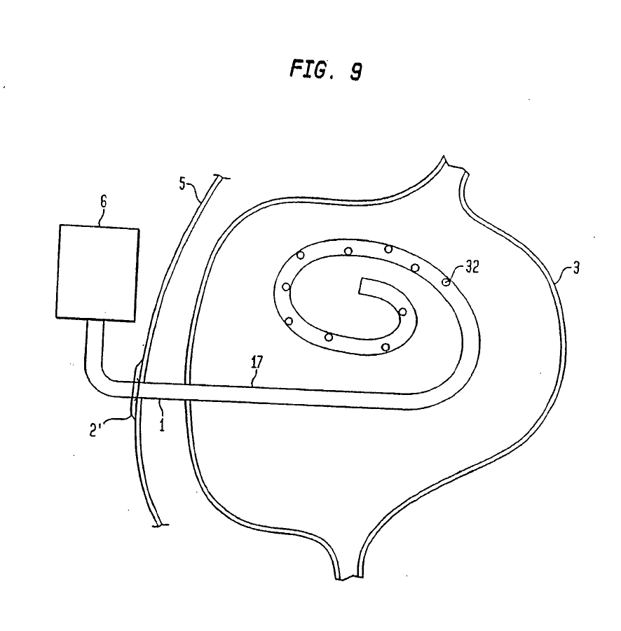

FIG. 8 illustrates a third embodiment of the present invention showing a tube having two balloons attached to that portion of the tube that is disposed within the patient's upper digestive system. The balloon anchor 2 is expandable to about 10 ml and is positioned up against the stomach wall to prevent the tube 1 from falling out. The inflatable balloon 29 is expandable from about 100 ml to about 850 ml and may be expanded intermittently to limit the capacity of the stomach. For example, the balloon 29 may be inflated via an inflation lumen prior to a meal to create the sensation of being full. After the meal, the balloon 29 may be deflated to prevent chronic accommodation. An electrically or a manually operated pump may be used to cause the inflation.

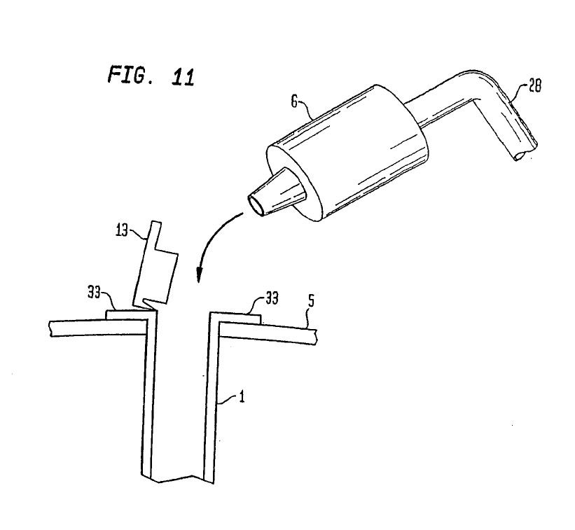

The tube 1 in this embodiment has a long inner tube length of about 10 cm or longer and a diameter of 28 French (9.3 mm) in size or greater. The tube 1 may have multiple holes 32 in the sidewall of its distal end portion 17 as shown in FIG. 8 and also in FIGS. 10 and 13-15B. The holes 32 may be 5×7 mm in size. The holes 32 provide non-vascular drainage from the patient. Preferably, the holes 32 are arranged in a spiral pattern 1 cm to 1.5 cm apart without losing structural integrity. More preferably, cushions or bumpers (not shown) are located on the tube 1 and in between the holes 32 to prevent the tube from being sucked up against the stomach wall while food is extracted from the upper digestive system out through the tube 1. For example, cushions or bumpers that are raised 3-4 mm above the surface of the tube 1 may be used for this purpose.

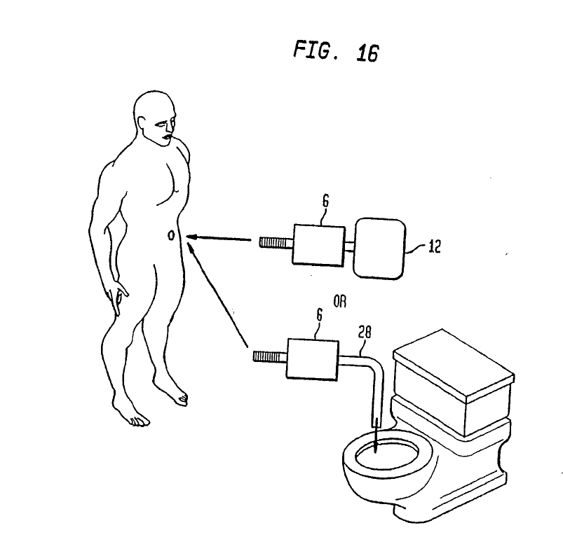

As shown in FIG. 8, a second retention member 33 may be attached at the proximal end portion 16 of the tube 1 to keep the tube 1 fixed to the abdominal surface. This second retention member may be similar to the retention members described hereinabove and shown in FIGS. 1, 1A, 1B and 6. The distance between the second retention member 33 at the proximal end portion 16 of the tube 1 and the balloon anchor 2 at the distal end portion 17 of the tube 1 can be adjusted to account for the varying amount of intervening tissue 40, 40′ as shown in FIGS. 15A and 15B. For example, the second retention member 33 may be attached to the tube 1 via an interference or friction fit. Specifically, the second retention member 33 may be placed around the outer surface of the proximal end portion 16 of the tube 1 and held in place on the tube 1 if it has an inner diameter that is slightly smaller than the outer diameter of the tube 1. As the patient loses weight, the proximal end portion 16 of the tube 1 extends farther and farther away from the patient's abdominal surface. A physician or the patient can slide the second retention member 33 down towards the abdominal surface and the excess amount of the tube 1 can be cut off.

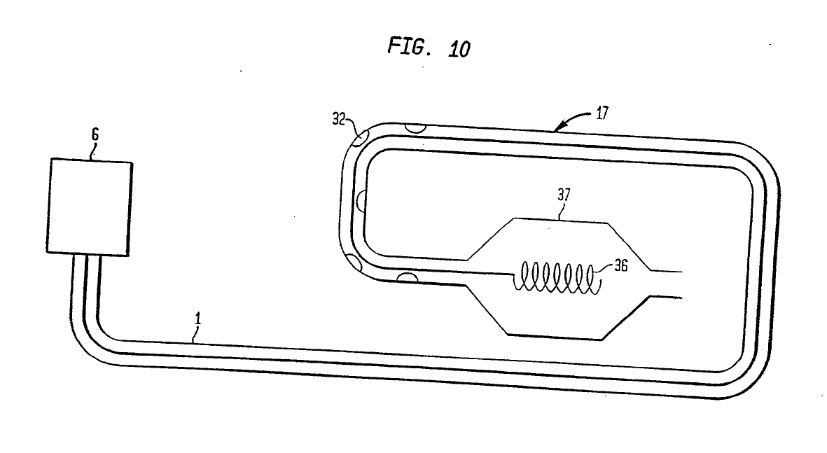

FIG. 9 illustrates a fourth embodiment of the present invention with a tube 1 having a curved configuration at its distal end portion 17 and a plurality of holes 32 in a sidewall. As shown in FIG. 9, the distal end portion 17 of the tube 1 is adapted to assume a curved configuration when disposed in the upper digestive system of a patient. Specifically, the distal end portion 17 of the tube 1 is flexible to facilitate insertion and removal from the patient. When the distal end portion 17 of the tube 1 is disposed in the upper digestive system of the patient, it returns to its natural curved configuration. The tube's tendency to return to its natural curved configuration may be achieved, for example, by bending the tube into a desired curved shape during the manufacturing process before the tube has fully cured or cooled, or by incorporating shape memory materials into the tube. As used herein, the term “curved” includes flexed, bent, rounded, arched, curled, coiled, spiral, and pigtail. This curved configuration is preferable because it increases the intake area within the upper digestive system. In addition, the coiled distal end portion 17 of the tube 1 as shown in FIG. 10 helps to maintain the position of the tube 1 within the patient's upper digestive system. The distal end portion 17 of the tube 1 may, for example, be about 10 cm long or longer to improve the intake of the food from the upper digestive system. Retention members (not shown) similar to the ones described in the above embodiments may also be used in this embodiment.

In an alternative embodiment (not shown), an actuating mechanism is configured to bend the distal end portion 17 of the tube 1 into a curved configuration. The actuating mechanism may, for example, be a string attached to the distal end portion 17 of the tube 1 that, when retracted causes the tube to assume a curved configuration (e.g. a loop with an arc that measures between about 270°-360°). A Cope Loop is a well known example of this arrangement.

FIG. 10 illustrates a fifth embodiment of the present invention showing a tube 1 having a curved configuration, multiple holes 32 in a sidewall, and a morcellation device 36 housed within a housing 37 at its distal end portion 17. Examples of morcellation devices are disclosed in U.S. Pat. Nos. 5,618,296, 5,741,287 and 5,520,634, herein incorporated by reference in their entirety. As shown in FIG. 10, a morcellation device 36 is provided at the distal end portion 17 of the tube 1 to divide and grind food into smaller pieces as it enters the tube 1. The morcellation device 36 thus allows large food to be removed from the patient without clogging the tube 1. The morcellation device 36 can be, for example, a mechanical propeller provided within a housing 37 at the distal end portion 17 of the tube 1. The housing 37 is constructed to protect body tissue from the morcellation device 36. In the illustrated embodiment, the housing 37 has an opening to permit the entry of food from the patient into the tube 1 and may, for example, be a cage that surrounds the morcellation device 36 at the distal end portion 17 of the tube 1. It is preferable that the housing 37 is collapsible in both directions so that it can be easily inserted into and taken out of the patient. The housing 37 is necessary to prevent damage to the stomach.

FIG. 11 illustrates a feature that may be used with any embodiment of the present invention in which the proximal end portion 16 of the tube 1 lies substantially flush with the outer surface of the patient's abdomen. This may be achieved by using ribbons attached to the tube 1, for example at the internal retention member. The ribbons are used to pull the tube 1 taut when the distal end portion 17 of the tube 1 is disposed in the upper digestive system of a patient. While the ribbons are pulled, the proximal end portion 16 of the tube 1 is cut so that the proximal end portion 16 lies flush with the abdominal surface and a thin, hollow cylinder with flanges is wedged onto the outside or inside surface of the tube 1 via friction or by screwing it onto the tube 1 to retain the tube 1 in its position and to keep it flush with the abdominal surface. In alternative embodiments, the proximal end portion 16 of the tube 1 may extend out past the abdominal surface by any desired length (e.g., 2-25 cm).

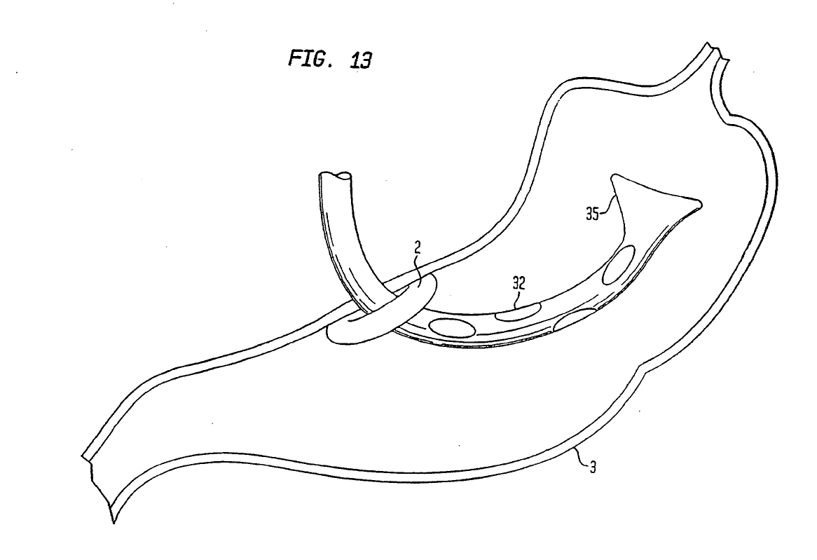

FIG. 12 illustrates another feature that may be used with any embodiment of the present invention in which a luer lock 34 is utilized at the proximal end portion 16 of the tube 1. In this embodiment, the pump 6 is attached to the tube 1 by screwing the pump 6 onto the tube 1 around the external portion of the proximal end portion 16 of the tube 1 rather than being inserted into the tube 1. More specifically, the proximal end portion 16 of the tube 1 comprises concentric grooves or threads on the outside to accommodate the pump 6, which prevents the pump 6 from reducing the size of the removal lumen 25. Likewise, the pump 6 may have corresponding concentric grooves or threads that allow it to interact and connect with the luer lock 34. In this way, large pieces of food can still be extracted out of the tube 1 because the inner diameter of the tube 1 is not compromised or decreased due to the pump 6 being inserted into the tube 1. Instead, the pump 6 is coupled to or threaded onto the outside of the proximal end portion 16 of the tube 1.

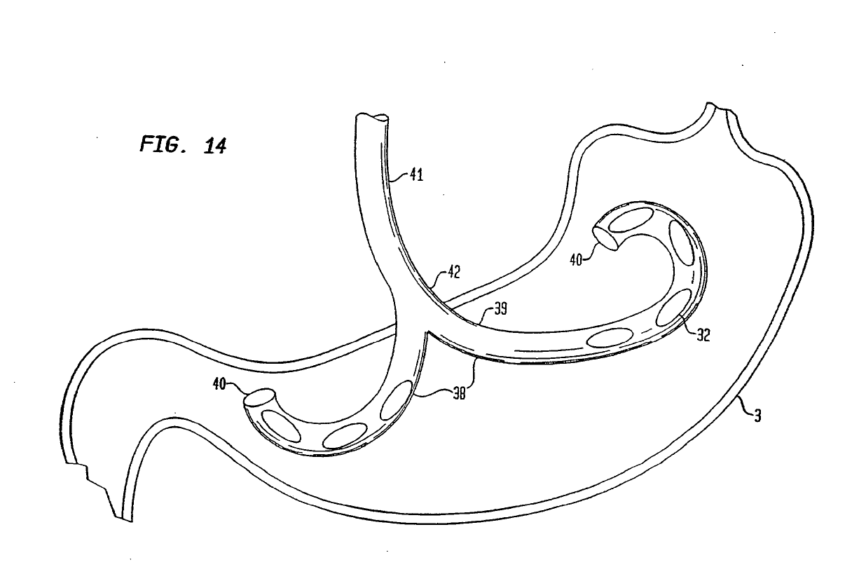

FIG. 13 illustrates yet another feature that may be used with any embodiment of the present invention in which the tube 1 has a funnel shaped tip 35. The funnel tip is advantageous because it facilitates the extraction of larger pieces of food into the tube 1 from the patient's digestive system.

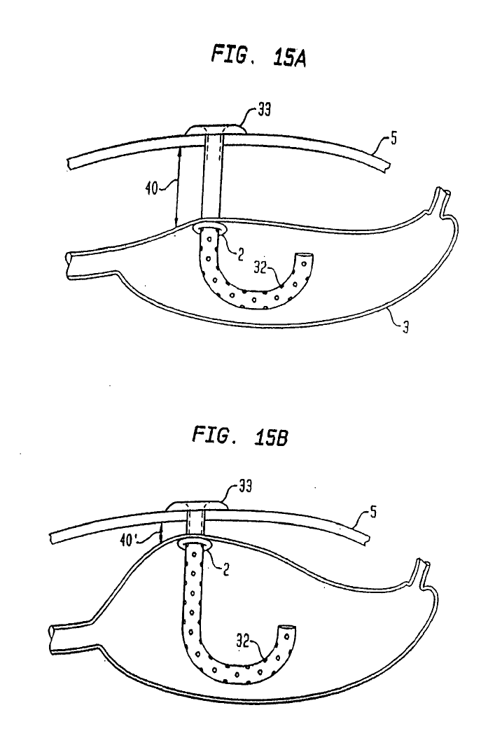

FIG. 14 illustrates a sixth embodiment of the present invention that has two intake tubes. In this embodiment, both of the intake tubes 38 have a curved configuration and a sidewall with a plurality of holes 32 located therein. Each intake tube 38 comprises a proximal end portion 39 and distal end portion 40. The apparatus also comprises an output tube 41 having a proximal end portion and a distal end portion 42. One or more retention members (not shown) are preferably attached to the output tube 41 to prevent the apparatus from coming out of the upper digestive system. The plurality of intake tubes 38 are configured to be disposed in the upper digestive system of the patient and the output tube 41 is configured to pass through the patient's abdominal wall when the plurality of intake tubes 38 are so disposed. The distal end portion 42 of the output tube 41 is operatively connected to the proximal end portion 39 of each of the plurality of intake tubes 38 so that food can be extracted from the upper digestive system of the patient through the distal end portion 40 of each of the plurality of intake tubes 38 and out through the proximal end portion of the output tube 41.

Optionally, pressure and/or flow sensors (not shown) may be placed on and/or in the tube 1. Pressure sensors placed on the tube 1 inside and outside the stomach 3 may be used to estimate the satiety of the patient. Alternatively or in addition to, flow sensors that are placed inside the tube 1 may be used to calculate the volume of food extracted through the tube 1.

Reference is now made to various methods for extracting food, for limiting absorption of food, and for treating obese patients.

Installation of any of the above-described embodiments forms a passageway into a patient's upper digestive system through the patient's abdominal wall. The patient is allowed to carry out his/her everyday affairs including ingesting food. After the patient has ingested food, the food is extracted by pumping it out of the upper digestive system through the passageway before it is completely digested. This method and the others described below are less invasive than the alternative surgical procedures for reducing weight, are easy to perform, easy to reverse and have successfully resulted in significant weight loss in obese patients.

In some methods, a tube is positioned so that it passes through a patient's abdominal wall into his/her upper digestive system. The patient is allowed to go about his/her daily activities including ingesting food. After the patient has ingested the food, the food is extracted from the upper digestive system of the patient through the tube. The patient may eat and extract the eaten food from his/her upper digestive system through the tube repeatedly until a desired weight loss is attained. The food that has been extracted is not reintroduced into the patient. The tube may be kept in the patient's upper digestive system for extended periods of time (e.g., one month or more) while the eating/extracting is repeated numerous times (e.g., 20 times or more) while the tube is in place.

In a second method, a tube is positioned so that it passes through the obese patient's abdominal wall into his/her upper digestive system. The obese patient is allowed to go about his/her daily activities including ingesting food. After the obese patient has ingested the food, the food is extracted from the upper digestive system of the obese patient through the tube. The obese patient may eat and extract the eaten food from his/her upper digestive system through the tube repeatedly until the obese patient has lost at least 40 pounds. The food that has been extracted is not reintroduced back into the obese patient.

In a third method, a tube is positioned so that it passes through a patient's abdominal wall into the upper digestive system of the patient whose gastrointestinal tract is unobstructed. The term “unobstructed,” as used herein, refers to a gastrointestinal tract that is not mechanically obstructed and is also not functionally obstructed. The patient is allowed to go about his/her daily activities including ingesting food. After the patient has ingested the food, the food is extracted from the upper digestive system of the patient through the tube. The patient may eat and extract the eaten food from his/her upper digestive system through the tube repeatedly until a desired weight loss is attained. The tube may be kept in the patient's upper digestive system for extended periods of time (e.g., one month or more) while the eating/extracting is repeated numerous times (e.g., 20 times or more) while the tube is in place.

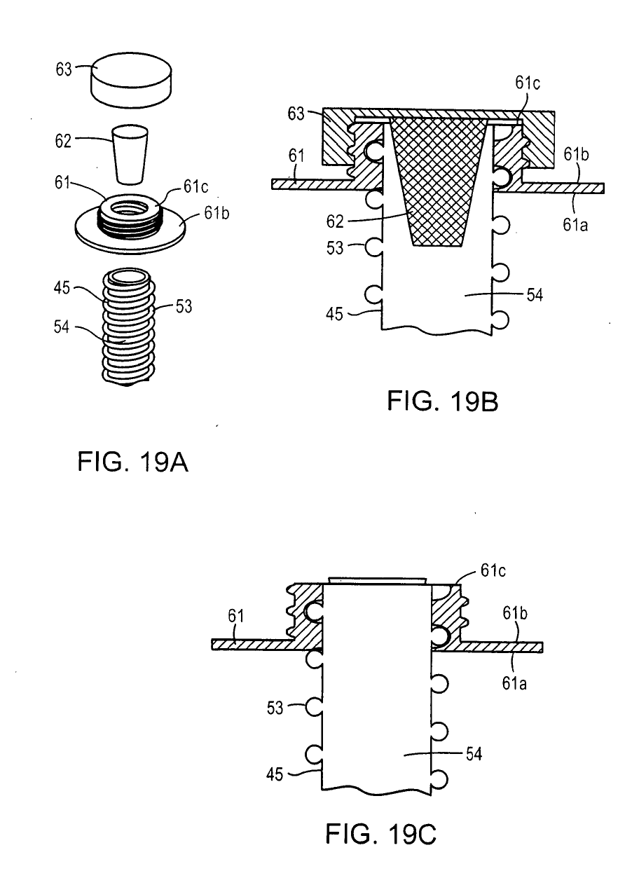

FIGS. 17A and 17B illustrate another embodiment of a tube 50 that can be used for food extraction or as a general gastrostomy device. More generally, the tube 50 can be placed in a body of a patient, e.g., within an organ or an anatomical space in the body of a patient. The anatomical space can be, for example, a gastrointestinal tract or within the stomach of a patient. In some embodiments, the tube 50 includes two primary segments: a proximal segment 45 and a distal segment 55, and the materials and dimensions of these segments are preferably selected to optimize performance of the tube 50. In some embodiments, the proximal segment 45 includes only a stoma tract segment 54. The proximal segment 45 can include the stoma tract segment 54 and also include additional tube length.

The stoma tract segment 54 has central lumen through which the food can be extracted. Although a large inner diameter (I.D.) is desirable to facilitate the extraction of food, the outer diameter should not be too large in view of the relevant anatomy. One suitable approach to balancing these opposing design characteristics is to form a tube 50 with a very thin wall, and to add a suitable external support structure, for example a helical support structure 53, to provide the necessary radial strength for the intended use. In addition, the stoma tract segment 54 of the tube 50 should be biocompatible. Since the stoma tract segment 54 is designed to span the length from the stomach, through the abdominal wall and reach the skin line in the patient. A suitable length in a patient will typically have a value of about 10 cm (although longer or shorter segments may be used if required for the anatomy of a particular patient). For example, a suitable length in an obese patient will typically have a value within a range of about 5 cm to about 15 cm. However, longer stoma tract segments may be required in a morbidly obese or a super morbidly obese patient and a shorter stoma tract segment may be needed for an overweight patient.

The inventors have determined that ePTFE (expanded polytetrafluoroethylene) is an excellent material for the proximal segment 45, the stoma tract segment 54, the distal segment 55, and/or the tube portion 51. The properties of ePTFE avoid fluid leakage at infusion pressures of less than approximately 9 psi, despite the ePTFE being microporous. In some embodiments, a tube 50 containing ePTFE has a water entry pressure measuring at least 4 psi. A suitable plastically deformable material yields at a lower pressure (e.g., 5 psi) and does not need a great deal of force, which enables use of simple tools to expand a portion of the plastically deformable material to enable it to provide a tight fit. A microporous material, for example a microporous plastically deformable material, could provide the same benefits as ePTFE without being expanded. Such a material could be, for example, a microporous PTFE. A typical microporous tube would leak at a much lower pressure (e.g., potentially a pressure lower than 2 psi) and not allow effective fluid infusion or drainage. One particularly suitable construction for the stoma tract segment 54 is to use a tube portion 51 made of ePTFE with an inner diameter having a measurement within the range of from about 5 mm to about 10 mm (e.g., about 7 mm). The tube portion 51 of the stoma tract segment 54 has a wall thickness having a measurement within the range of from about ¼ mm to about 2 mm (e.g., about ½ mm). The tube portion 51 wall thickness is reinforced by a helical structure 53 that is affixed to at least a portion of the outside surface of the tube portion 51. In some embodiments (not shown) the helical structure 53 extends beyond the tube portion 51 of the stoma tract segment 54 to support at least some of the distal portion 55 of the tube 50. In some embodiments, the tube 50 has an internal diameter that is 1 mm smaller than its outer diameter.

ePTFE materials have a range of internodal distances. A segment of ePTFE material having small internodal distances avoid liquids from entering the tube, and also, such a material elicits biological incorporation of the material into the patient. The ePTFE material internodal distance is selected to achieve a desired biological incorporation of the tube 50 in the body of the patient. The ePTFE internodal distance can be between about 10 μm and about 120 μm. For example, in some embodiments, the tube portion 51 is made from ePTFE having an internodal distance within the range of from about 10 μm to about 120 μm. Optionally, different internodal distances may be used for different portions of the tube portion 51 to optimize the properties to the application at hand. For example, a higher internodal distance (e.g. 40 μm to 60 μm) may be provided on the outer diameter to facilitate biological incorporation and a smaller internodal distance (e.g. on the order of 5 μm) may be used on the luminal (inner) surface to decrease the permeability to fluids and gases. In some embodiments, the average distance between nodes varies along the length of at least a portion of the tube 50, for example.

A conventional PEG sized at 28 French is the largest conventional PEG that has a corresponding 6.3 mm inner diameter. A goal of a design employing ePTFE for the proximal segment 45 is to maximize the inner diameter while minimizing the outer diameter. For example, to make a greater than 6.3 mm inner diameter with an outer diameter that is smaller than 28 French. In some embodiments, the inner diameter is 6.3 mm or greater and the tube resists collapse when extraction is performed. For example, optionally, the inner diameter is 6.5 mm.

A variety of materials and configurations may be employed to form a helical structure 53 for the above-described configuration, as will be apparent to persons skilled in the relevant art. In particular, the inventors have determined that PTFE (polytetrafluoroethylene) is an excellent material for the helical support structure 53. Suitable helical structures 53 are formed from, for example, a helical bead of PTFE. Alternatively, a Kevlar braid may be used in place of the PTFE helical bead. In some embodiments, the tube portion 51 of the stoma tract segment 54 is a thin-wall silicone or polyurethane tube and the helical support structure 53 that externally supports the tube portion 51 is a wire coil for example, a stainless steel wire coil. Optionally, an elastomer is molded in tandem with a wire coil to incorporate the wire into the wall of the tube portion 51. In some embodiments, one or more of an PTFE bead, a Kevlar braid, and a wire coil are employed as a support structure of a stoma tract segment 54. The helical support structure 53 may be attached to the tube portion 51 by mechanical bonding, by chemical bonding (e.g., thermal bonding), or by any of a variety of other bonding approaches that will be apparent to persons skilled in the relevant art.

A suitable diameter of the material that forms the helical support structure 53 has a measurement within the range of from about ¼ mm to about 1 mm (e.g., about ½ mm). A suitable helical pitch for the helical support structure 53 is on the order of about 2-3 mm. One or more of the materials used to form the helical support structure 53, the diameter of the helical support structure 53, and/or the helical pitch of the helical support structure 53 are selected to provide a desired radial strength of the portion of the tube 50 surrounded by the helical support structure 53. The helical support structure 53 may be designed to provide one or more of a desired radial strength, a desired flexibility, and a desired kink-resistance.

In an embodiment where the tube portion 51 is made from ePTFE the microporous nature of the ePTFE stoma tract segment 54 has been shown to elicit biological incorporation when implanted in animal study models. This biological incorporation, especially around the stoma tract segment 54 entry into the stomach, advantageously improves the stability of the stoma tract segment 54 interface with the stomach, reduces trauma to the stomach upon external tube movement and improves the seal to fluid-flow around the stoma tract segment 54 (which reduces the potential for leakage of gastric contents). In an animal model, compared to a standard silicone PEG tube the ePTFE stoma tract segment 54 of the tube 50 also showed the ability to reduce granulation tissue formation around the skin exit site. In alternative embodiments, microporous materials other than ePTFE may be used.

Using this arrangement of materials with a tube portion 51 for the stoma tract segment 54 and a helical support structure 53 on the outside surface of at least a portion of the tube 50 advantageously provides a smooth inner surface and maximizes the inner diameter without unduly increasing the outer diameter. The design incorporating a helical support structure 53 disposed on an outside surface of at least a portion of the tube 50 also provides radial strength to prevent tube collapse, provides superior flexibility and kink-resistance.

A distal segment 55 is provided distal to the proximal segment 45, so that the lumens of those two segments 45, 55 cooperate to form a fluid path. A suitable length for the distal segment 55 has a measurement of about 15 cm. The length of the distal segment 55 can vary depending upon the size of the patient or the size of a patient's stomach. In some embodiments, the inner diameter of the distal segment 55 is the same as the inner diameter of the stoma tract segment 54 of the proximal segment 45. Alternatively, the inner diameter of the distal segment 55 is different from the inner diameter of the stoma tract segment 54 of the proximal segment 45. In some embodiments (not shown), the distal segment 55 is made of the same materials and has the same construction as the stoma tract segment 54. Alternatively, the distal segment 55 is made from a different material than the stoma tract segment 54. Since ePTFE and PTFE are relatively expensive materials and because the characteristics of the distal segment 55 are less critical (the distal tube segment 55 does not require biological incorporation) costs can be reduced by using a less expensive material to form the distal segment 55. Suitable less costly materials for use in the distal segment 55 include silicone, polyurethane, or other medical grade elastomers or flexible polymers (e.g. low density polyethylene). When silicone is used, a wall thickness of about 1.5 mm is suitable to provide mechanical strength, resulting in an outer diameter of about 30 French.

The distal segment 55 may be connected directly to the distal end of the stoma tract segment 54 (e.g., where the proximal segment 45 includes the stoma tract segment 54 and no additional tube length) to permit fluid flow therebetween by, for example, molding those two segments together by mechanical techniques (e.g., tension fit) or chemical techniques (e.g., using a suitable adhesive or using heat). Alternatively, intervening components, for example, connectors (not shown) may be interposed between the distal segment 55 and the stoma tract segment 54. In some embodiments, the distal segment 55 has at least one intake hole through which food can enter. In the illustrated embodiment, the distal segment 55 has multiple holes 56 located in the sidewall of the distal segment 55 arranged in a spiral pattern. Alternatively, multiple holes 56 may be disposed through the sidewall of the distal tube segment 55 in a random fashion or according another suitable design. Suitable holes 56 have a size and spacing that does not adversely impact structural integrity of the distal segment 55. For example, a suitable size and spacing for the holes in, for example, a tube having a 7 mm inner diameter that does not adversely impact structural integrity while allowing particle aspiration and preventing tube clogging is to use holes 56 that measure less than or equal to 6×8 mm and are spaced between about 1.5 cm and about 3 cm apart.

In some embodiments, a retention member is provided between the distal segment 55 and the proximal segment 45, the retention member can be, for example, a bumper 59. Generally, the retention member is coupled to a tube 50 to prevent dislodgement of the tube 50 to the exterior of a patient's body. In some embodiments, the retention member prevents dislodgement of the tube 50 to the exterior of a patient's body without an exerted force. The bumper 59 is preferably dimensioned and configured such that when the tube 50 is implanted in the stomach of a patient the bumper 59 butts up against the inside wall of the stomach. The bumper 59 is preferably dimensioned and configured to prevent the gastrostomy tube 50 from being inadvertently pulled out of the stomach, while simultaneously allowing a physician to remove the device using manual traction. For example, in some embodiments a dome bumper 59 has a 2.5 cm diameter, 1 cm height, and a 1.25 cm wall thickness. Suitable materials for the bumper 59 include silicone and polyurethane, and a suitable construction for the bumper 59 include domed bumpers such as the domed bumper 59 used in the Bard Ponsky™ PEG Tube. The bumper 59 may be attached to the distal segment 55 and stoma tract segment 54 using any appropriate method, including, but not limited to, molding those components together or using an appropriate adhesive.

Referring still to FIGS. 17A and 17B, during use of the tube 50 inside the body of a patient, the bumper 59 rests inside the stomach and is butted up against the stomach wall. The stoma tract segment 54 passes through the stomach wall to the outside world and the distal segment 55 is disposed in the body of the patient such that it rests inside the stomach. When the patient consumes food, the food is drawn into the holes 56 disposed through the distal segment 55, the food travels through the distal segment 55, continues through the stoma tract segment 54, and exits the body of the patient through the opening in the stoma tract segment 54.

Optionally, a compliant hydrophobic washer 52 may be affixed to the mucosa contacting surface of the retention member, bumper 59. The preferred material for a washer 52 is ePTFE. Suitable washers are sized from about 1 cm to about 3 cm (e.g., about 2.5 cm) in diameter and measure from between about ⅛ to about ½ mm thick. In an embodiment when the washer 52 is compressed against the mucosa of the stomach wall by the retention member 59 in cooperation with an external retaining mechanism (see, FIG. 1 flange 2′), the mechanical properties of the washer 52 may aide in sealing gastric fluids from leaking around the tube 50.

Making a portion of the tube 50 (e.g., tube portion 51) from ePTFE provides a number of advantages compared to silicone tubes, including: (a) improved tissue healing; (b) greater resistance to bacteria colonization; (c) greater flexibility and kink resistance, which reduces the stress exerted on the stoma tract and stomach entry site, and reduces the risk of leakage and tissue inflammation; and (d) a more lubricious luminal surface, which permits food to move through the tube more freely. ePTFE is also one of the most inert synthetic polymers, which is useful for resistance to degradation by stomach acids and to minimize any inflammatory response by surrounding tissue. Although ePTFE construction is expensive, the described benefits will often justify the added cost particularly for active patients and obese patients who require gastrostomy tubes for extended durations.

Referring still to FIG. 17A-17B, in some embodiments, the tube 50 has a proximal segment 45 that is configured to pass through the patient's skin when the distal segment 55 is disposed in the body of a patient, e.g., in a patient's organ. A helical support 53 is disposed on an outside surface of at least a portion of the tube 50. The helical support 53 can be disposed on the proximal segment 45, for example, about the outside surface of the stoma tract segment 54. At least a portion of the proximal segment 45 can be a microporous structure such as, for example, ePTFE. Optionally, the distal segment 55 has a plurality of holes 56 located in its sidewall.

In some embodiments, the tube 50 has a lumen and at least a portion of the tube 50 is configured to pass through a patient's skin. In some embodiment, the portion of the tube 50 that passes through the patient's skin has a microporous structure (e.g., ePTFE) that does not leak when liquid flows through the lumen at a pressure of less than 4 psi. The microporous structure can have an internodal distance of from about 5 μm to about 120 μm, for example. The internodal distance can be selected to achieve a desired biological incorporation of the tube 50 in the patient's skin, body, or abdominal wall, for example. A helical support 53 can be disposed on the portion of the tube 50 that passes through the patient's skin. The portion of the tube 50 that passes through the patient's skin can be a proximal segment 45 including, for example, the stoma tract segment 54.

In some embodiments, the tube 50 has a distal segment 55 and a proximal segment 45 and the proximal segment is configured to pass through the patient's abdominal wall when the distal segment is disposed in the patient's upper digestive system. The tube 50 has a distal segment 55 can be made from a variety of materials including, for example, an elastomeric extruded material (e.g., silicon). The distal segment 55 has a wall thickness. The proximal segment 45 of the tube 50 can include a material different from the distal segment 55. The proximal segment 45 can have a proximal segment wall thickness measuring at least 25% less than the distal segment wall thickness.

For example, in one embodiment, the distal tube 55 has an outer diameter that is 28 French (9.3 mm) in size and an inner diameter that measures 6.3 mm and the proximal segment 45 has an outer diameter that is 24 French (8.0 mm) in size and an inner diameter that measures 7 mm. Thus, the distal segment 55 wall thickness is about 1.5 mm and the proximal segment 45 wall thickness is about 0.5 mm, thus the proximal segment 45 has a wall thickness that is about 67% less thick than the distal segment 55 wall thickness.

In another embodiment, the distal tube segment 55 has a larger outer diameter than the proximal segment 45, but both segments 55,45 of the tube 50 have the same inner diameter. More specifically, the distal tube 55 has an outer diameter that is 28 French (9.3 mm) in size and an inner diameter that measures 7 mm and the proximal segment 45 has an outer diameter that is 24 French (8.0 mm) in size and an inner diameter that measures 7 mm. Thus, the distal segment 55 wall thickness is about 1.15 mm and the proximal segment 45 wall thickness is about 0.5 mm, thus the proximal segment 45 has a wall thickness that is about 57% less thick than the distal segment 55 wall thickness. The proximal segment 45 wall thickness can measure at least about 20%, at least about 40%, at least about 60%, or at least about 80% less than the distal segment 55 wall thickness.

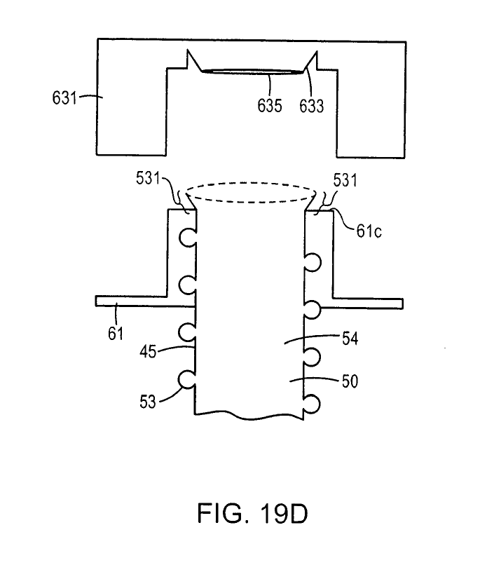

FIG. 18 shows a suitable mechanism for maneuvering the gastrostomy tube 50 into position with the washer (or the bumper 59 if the washer is omitted) butted up against the stomach wall. A long proximal leader 57 (e.g., a 50 cm long, 21 F silicone tube with an inner diameter of about 2.5 mm) is attached to the proximal end 161 of the proximal segment 45, which is also proximal end of the stoma tract segment 54. The leader 57 may be attached by the original manufacturer, but may also be attached subsequently (e.g., by the doctor just prior to insertion of the gastrostomy tube 50). Suitable approaches for attaching the leader 57 to the stoma tract segment 54 include adhesives, shrink-tubing, etc. Optionally, a tapered dilator 58 may be provided at the proximal end of the leader 57, e.g., using an interference fit, shrink-tubing, and/or adhesive bonding to hold a 4 mm diameter protruding post (not shown) at the distal end of the dilator 58 into the leader 57. The leader 57 has an inner diameter measuring about 2.5 mm. A pull wire 60 may then be attached to the dilator 58, and used to pull the leader 57 and the attached gastrostomy tube 50 down the patient's esophagus and out through an incision into the stomach, as described above, until the proximal end 161 of the stoma tract segment 54 extends out through the incision and the bumper 59 (or where employed a washer 52) hits the inner wall of the stomach. Of course, alternative delivery approaches may also be used, which will be apparent to persons skilled in the relevant arts.

After the gastrostomy tube 50 is so positioned, the leader 57 is cut off (at a point distal to the interface between the leader 57 and the stoma tract segment 54) and discarded. At this point, the gastrostomy tube 50 depicted in FIG. 17 remains, with the bumper 59 optionally, a washer 52, and the distal segment 55 located inside the patient's stomach and with the stoma tract segment 54 passing through and protruding out from the patient's abdominal wall. It must then be terminated so that it stays in place in the patient's body.

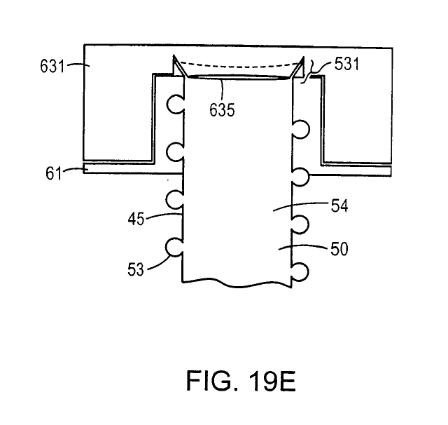

FIGS. 19A-C depict a first set of components used to create a low-profile termination for the stoma tract segment 54 such that when the tube 50 is placed in a patient the stoma tract segment 54 is preferably as flush as possible with the skin on the surface of the patient's outer abdominal wall. FIG. 19A shows these components in an exploded view, including the proximal end of the proximal segment 45, the stoma tract segment 54, a flange 61, a stopper 62, and a cap 63. The flange 61 preferably has internal thread that complements and is dimensioned to mate with the helical support structure 53 disposed about the outside surface of the stoma tract segment 54. The cap 63 removably mates with the flange 61, for example with an external thread on the flange 61.

As best seen in FIG. 19B (which is a cross section of the a flange 61 mounted onto the stoma tract segment 54), after the gastrostomy tube 50 is positioned in the patient with the stoma tract segment 54 positioned through the patient's abdominal wall as described above, the flange 61 is screwed onto the threaded configuration of the helical support structure 53 of the stoma tract segment 54 until the lower surface 61a of the flange 61 hits the skin-line. In this fashion, the internal threads of the flange 61 hold the stoma tract segment 54 so that the bumper 59 or the washer 52 (shown in FIGS. 17A-17B) is urged against the inner wall of the patient's stomach. The portion of the stoma tract segment 54 that protrudes above the top of the flange 61 is then cut off and discarded. This screw-type adjustment of the flange 61 with respect to the stoma tract segment 54 (by clockwise or counter-clockwise rotation) provides fine control over the length of the stoma tract segment 54 that resides in the abdominal wall after insertion/placement of the tube 50 in the patient. In addition, when the thickness of the patient's abdominal wall shrinks (due to weight loss), that length of the stoma tract segment 54 can be easily re-adjusted by screwing the flange 61 down some more on the stoma tract segment 54. After such a re-adjustment of the stoma tract segment 54 the portion of the stoma tract segment 54 that protrudes above the top surface 61c of the flange 61 is cut off and is discarded. Optionally, the flange 61 is configured with respect to the stoma tract segment 54 of the proximal segment 45, an inside surface of the flange 61 is adapted to mate with a portion of the proximal segment 45 such that a portion of the flange 61 will lie substantially flush with the patient's abdominal surface when the distal segment 55 of the tube 50 is disposed in the upper digestive system of the patient. In an alternate embodiment, the cap 63 has indentations within the flange 61 and the cap 63 removably mates with the flange 61. The stoma tract segment 54 of the tube 50 could have discrete transverse rings or protrusions disposed on the proximal segment 45 of the tube 50 that ratchet through any complementary indentations within the inner through-hole of the flange 61. In such a fashion, the tube 50 can be pushed down flush above the skin-line and fixed in place at the nearest detent.

In FIGS. 19A-19B, the flange 61 is also shown to have external threads onto which the cap 63 can be screwed. The external threads are disposed between the top surface 61c and the middle surface 61b of the flange 61. The cap 63 has corresponding internal threads dimensioned to removably mate with the external threads of the flange 61. Although a seal of the stoma tract segment 54 may be provided by the interaction between the external threads of the flange 61 and the internal threads of the cap 63 alone, the seal may optionally be improved by using a stopper 62 to seal the end of the stoma tract segment 54 to reduce the amount of cleaning that will be required. FIG. 19B shows, in cross section, the interaction between the various components when such a stopper 62 is used when (a) the flange 61 is screwed onto the stoma tract segment 54, (b) the stopper 62 is inserted into the stoma tract segment 54, and (c) the cap 63 is screwed onto the flange 61. This arrangement serves to seal the lumen of the stoma tract segment 54 from the outside world with a fluid-tight seal. To allow fluid communication via the gastrostomy tube 50, the cap 63 and stopper 62 are removed and an external tube or pump (see, e.g., FIGS. 2-4 and 11) is connected to the flange 61.

Although FIGS. 19A-19C depict one particular mechanism for alternately providing an opening and a fluid-tight seal, persons skilled in the relevant arts will recognize that a wide variety of alternative approaches may be used in place thereof. Examples include a cap that has a tension fit with an exterior of a portion of the flange, a cap that has a tension fit with an interior of a portion of the flange, screw-on caps, snap-on caps, stoppers, magnetically attached caps, etc. In any of the above cases, an attachment mechanism (e.g., a hinge, a tether, etc.) may be used to prevent the cap from getting lost. Alternatively, instead of using a cap 63, a valve may be used to mate with the flange 61 the valve may include an actuator (e.g., slide, rotary, toggle, push-button, etc.) that permits the user to open or close the fluid path of the tube 50, as desired. The valve prevents and allows fluid flow into and out of the tube 50.

An additional feature of the stoma tract segment 54 is that it can be plastically deformed to increase its diameter. The plastically deformable stoma tract segment 54 can be permanently stretched to a larger diameter by using a mechanism that provides internal radial force. For example, the stoma tract segment 54 diameter can be increased by using, for example, a radially expanding mandrel, an inflatable bladder, a balloon, or a dilator. At least a portion of the stoma tract segment 54 includes a microporous material that is plastically deformable. Referring now to FIG. 19D, in some embodiments, at least a portion 531 of the stoma tract segment 54 contains ePTFE, which plastically deforms at low intraluminal pressure (e.g., intraluminal pressures having a value of from about 15 psi to about 30 psi). ePTFE is plastically deformable, a characteristic that differs from elastic deformation that enables the ePTFE material to retain its placement in a patient's body.

To avoid tube 50 leakage it is desirable to create a fluid-tight seal between the tube 50 placed in a patient and the exterior of the patient's body. In some embodiments, a fluid-tight seal is created between the stoma tract segment 54, the flange 61, and a cap 631.

Increased strength of attachment of the stoma tract segment 54 to the flange 61 can be created by dilating a portion 531 of the stoma tract segment 54 over an approximately 2-mm length above the flange 61. The dilated portion 531 of the stoma tract segment 54 allows insertion of a cap tube 633 into the dilated portion 531 of the stoma tract segment 54. The cap tube 633 has a thru hole 635 that has substantially the same internal diameter as the internal diameter of the portion of the stoma tract segment 54 other than the dilated portion 531. In some embodiments, the cap tube 633 is attached to cap 631. Alternatively, the cap tube 633 is attached to a face plate or to a portion of an assembly that creates a cap 630 or other termination of the stoma tract segment 54. In some embodiments, the cap tube 633 is attached to a valve that provides controlled access to the stoma tract segment 54.

In some embodiments, the cap 631 together with the cap tube 633 is mechanically coupled to the flange 61. Referring also to FIG. 19D, mechanical coupling of the cap 631 with the flange 61 is accomplished by any conventional fastening means (e.g. snap-fit, threaded fit, and tension fit). When the cap 631 together with the attached cap tube 633 is fastened to the flange 61 the cap tube 633 enters the stoma tract segment 54 and a portion of the stoma tract segment 54 is sandwiched between the cap tube 633 and the inside surface of the flange 61. In some embodiments, the dilated portion 531 of the stoma tract segment 54 enables entry of the cap tube 633 into the stoma tract segment 54.

The sandwiched portion of the stoma tract segment 54 creates a fluid-tight seal between the flange 61 and the stoma tract segment 54. The material in the sandwiched portion has properties that enable creation of the fluid-tight seal and such properties include, for example, it is hydrophobic, plastically deformable, and provides mechanical compliance that enables crevices between the inside surface of the flange 61 and stoma tract segment 54 to be filled. ePTFE has hydrophobic properties and mechanical compliance that enable filling of crevices and creation of a fluid-tight seal between the stoma tract segment 54 and the flange 61 and/or the cap 633.

In addition, the portion of the stoma tract segment 54 sandwiched between the cap tube 633 and the inside surface of the flange 61 is mechanically clamped between the flange 61 and the cap tube 633 to create a strong mechanical attachment. Additionally, the radial expansion of the ePTFE tube end (i.e., the dilated portion 531) allows a cap tube 633 having a through hole 635 internal diameter that matches the diameter of the portions of the stoma tract segment 54 other than the dilated portion 531 diameter to be inserted in the stoma tract segment 54. Thus the dilated portion 531 enables a consistent lumen dimension (i.e., inner diameter) throughout the entire stoma tract segment 54, lumen 61, and cap 631 assembly shown in FIG. 19E.

The resistance to deformation in response to a radial force that is provided by the ePTFE material and/or the helical structure 53 avoid tube restriction that can create resistance to aspiration. The cap 631, flange 61, and stoma tract segment 54 enable retrofit/customization upon patient weight loss. For example, the cap 631 and the cap tube 633 can be detached from the flange 61, to allow placement of the flange 61 in closer apposition to the skin-line and shortening of the stoma tract segment 54. For example, when the cap 631 is removed from the flange 61 (see, FIG. 19D) the threaded inner diameter of the flange 61 is twisted over the helical structure 53 in closer proximity to the skin-line of the patient. A portion of the stoma tract segment 54 that protrudes above the top surface 61c of the flange 61 is removed, e.g., the protruding portion is cut off and is discarded. A portion of the stoma tract segment 54 can be dilated, as discussed above, and thereafter the cap 631 can be reattached to the flange 61, such that the cap tube 633 enters the stoma tract segment 54 to reintroduce fixation and a fluid tight seal between the tube 50, the flange 61, and the cap 631.

Referring now to FIGS. 19A-19E, in some embodiments, a kit for use in the body of a patient includes a low-profile termination for a gastrostomy tube. For example, a suitable kit can include a gastrostomy tube having a helical support 53 disposed on at least a portion of an outside surface of the gastrostomy tube, a flange 61, and a cap 63, 631. An inside surface of the flange 61 has a thread that complements the helical support 53 such that when the flange 61 is screwed down onto the tube, a portion of the flange 61 lies substantially flush with an exterior surface of a patient's skin. The cap 63, 631 detachably couples to the flange 61. The cap 63, 631 can have an inside surface with internal threads dimensioned to mate with external threads disposed on an outside surface of the flange 61. The cap 63, 631 can include a valve that prevents and allows fluid flow into and out of the gastrostomy tube. The flange 61 can have a low profile (i.e., not protrude off of the patient's skin by more than about 2 cm or by more than about 1 cm). The tube can be made from a plastically deformable material. The flange 61 has a thread that interacts with the helical structure 53 to adjust the tube length by exposing a portion of the tube exterior to the flange 61 and the portion of the tube exterior to the flange 61 can be detached by cutting. In some embodiments, a kit is assembled for use with a gastrostomy tube having a helical support 53 disposed on at least a portion of an outside surface of the gastrostomy tube. The kit includes flange 61 and a cap 63, 631. The flange 61 has an inside surface with a thread that complements the helical support 53 such that when the flange 61 is screwed down onto the tube, a portion of the flange 61 lies substantially flush with an exterior surface of a patient's skin and a cap 63, 631 detachably couples to the flange 61.

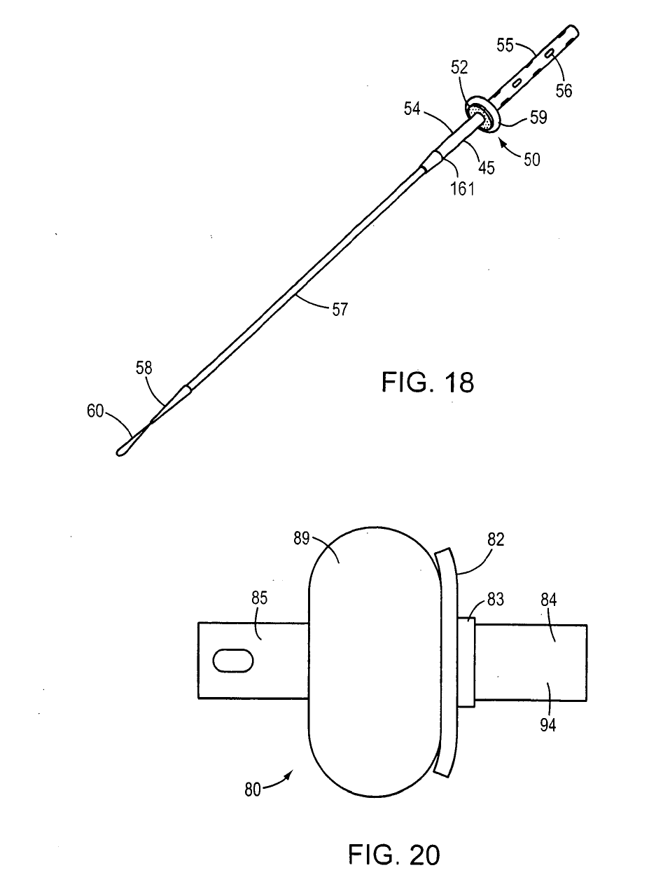

FIG. 20 depicts an alternative gastrostomy tube 80 that is much less expensive than the tube 50 shown in FIGS. 17A-17C, yet the gastrostomy tube 80 retains some of the advantages of the tube 50 by making limited use of ePTFE. In the embodiment shown in FIG. 20, the distal segment 85 is similar to the distal segment 55 of FIGS. 17A-17C, and the bumper 89 is similar to the bumper 59 discussed in relation to FIGS. 17A-17C. In some embodiments, the bumper 89 is disposed between the distal segment 85 and the proximal segment 94. However, instead of using ePTFE for the complete stoma tract segment 54 in FIG. 20 at least a portion of the proximal segment 94 includes ePTFE. For example, a length of the proximal segment 94 includes ePTFE and the remaining portion of the proximal segment 94 is made from other materials, for example, silicon PEG or thick-walled silicon. The length of ePTFE in the proximal segment 94 ranges from about ½ cm to about 1 cm.

In some embodiments, a thick-walled silicon tube 84 is used, e.g., with an inner diameter of about 6 mm and an outer diameter of about 28 F at least a portion of the proximal tube segment 94 includes ePTFE. In some embodiments, a tubular sleeve including ePTFE is configured to surround the outer diameter of at least a portion of the proximal segment 94 of the tube 80. For example, in some embodiments, the tubular sleeve is an ePTFE collar 83 that fits over the silicone tube 84 proximal to the bumper 89. A suitable length for the collar 83 ranges from about ½ cm to about 1 cm. In this embodiment, the standard properties of a silicone PEG tube remain, with the added benefit of biological incorporation of the stoma tract segment 94 into the ePTFE collar 83 near and through the patient's stomach wall. Thick-wall type silicone PEG tubes are preferred to provide sufficient radial strength and kink-resistance. The flexibility of thick-wall silicone is not great and the inner diameter of the tube 80 is restricted to approximately 6 mm, however, a tube 80 having such a construction will still function acceptably. Note that using diameters larger than 28 F for the stoma tract segment 94 can increase the risk of complications, so appropriate precautions should be taken.

Optionally, the ePTFE collar 83 may be configured so that it can slide on the silicone tube 84 with little friction, such that external forces on the tube 80 allow the bumper 89 to move further into the patients stomach without causing trauma on the biological interface at the level of the stomach wall and in the adjacent stoma tract.

In an alternative embodiment, the tubular sleeve is an ultrathin (e.g., about 0.05 mm thick) ePTFE sleeve that is placed over a standard 28 F silicone PEG tube 84 to maintain the standard silicone PEG tube mechanical properties while allowing biological incorporation into the stoma tract segment 94. In an alternative embodiment, the proximal segment 94 is a composite tube (e.g., the composite tube features braiding with a metallic or polymer fiber or ribbon, or by wrapping the exterior of a thin walled PEG tube, a standard silicon PEG tube, or a PEG tube with an ePTFE sleeve with a metal or polymer fiber or ribbon) may be used to achieve an inner diameter greater than 6 mm while maintaining an outer diameter of less than or equal to 28 F, with mechanical properties equal or superior to the thick-wall silicone tube.

In any of the above-described embodiments, referring now to FIG. 20, an ePTFE washer 82 may optionally be positioned between the bumper 89 and the silicon tube 84 so that when the gastrostomy tube 80 is installed in the patient's body, the ePTFE washer 82 rests against the inside of the patient's stomach wall, with the bumper 89 and the distal 85 located inside the patient's stomach, and the stoma tract segment 94 passing through and out of the patient's abdominal wall.

In some embodiments, a helical support structure described in relation to FIGS. 17A and 17B is disposed on an outside surface of at least a portion of the tube 80. For example, the helical support structure may be disposed about at least a portion of a tubular sleeve. Features described in relation to tube 50 (FIGS. 17A-17B), the low-profile termination (FIGS. 19A-19C), and the installation mechanism (FIG. 18) may be employed in association with the tube 80 (FIG. 20).

Preliminary trials in human patients have been successful. For example, one female patient, middle aged and weighing 100 kilograms (approximately 220 pounds), had a tube installed in her stomach for 59 weeks and successfully lost 38.45 kilograms (approximately 85 pounds) without experiencing any serious adverse side effects. During the 59 weeks, the female patient aspirated after breakfast and lunch meals daily. She consumed meals without any fluids over approximately 30 minutes. At the end of the meal, she consumed 52 ounces of water in approximately 3-4 minutes. She waited approximately 20 minutes after consuming the water before beginning the extraction procedure. Accordingly, the patient uncapped the tube, connected a 60 cc syringe to the tube and extracted food from her stomach twice. This resulted in a siphon effect, which permitted the subject to freely drain the stomach by allowing the open tube to empty into a bucket. The patient squeezed the tube to enhance propulsion and to break up large food. After draining stopped, the patient usually drank another 52 ounces of water and repeated the extraction procedure. She usually repeated this procedure (drinking and extracting) about 2 more times, until she felt her stomach was empty. The total amount of food extracted was approximately 2-3 liters and the entire procedure took about 20 minutes. If resistance to extraction occurred during the procedure, the patient flushed the tube with 30 cc of water. The water helped to extract the food by dissolving it and by cleaning the passageway. The patient changed her dietary intake to avoid tube clogging. She avoided eating cauliflower, broccoli, Chinese food, stir fry, snow peas, pretzels, chips, and steak. In addition, her diet was supplemented with potassium. The chart below illustrates her weight loss.

| | weight |
|---|---|
| Week | (kg) |
| 0 | 100.9 |
| 2 | 96.8 |
| 3 | 96.8 |
| 4 | 94.7 |
| 4 | 94.7 |
| 5 | 94.0 |
| 7 | 93.6 |
| 8 | 90.9 |
| 9 | 92.9 |
| 10 | 92.7 |
| 11 | 90.4 |
| 12 | 89 |
| 13 | 89.3 |
| 14 | 88.6 |
| 15 | 87.7 |
| 16 | 86.5 |
| 17 | 86.5 |
| 18 | 86.3 |
| 19 | 85.9 |
| 20 | 83.9 |
| 21 | 82.9 |
| 22 | 81.6 |
| 23 | 80.45 |
| 24 | 79.7 |
| 25 | 78.6 |
| 26 | 78.6 |
| 27 | 77.2 |
| 28 | 78 |
| 29 | 76.2 |
| 30 | 76 |
| 31 | 75.2 |
| 31 | 77.1 |
| 32 | 76.4 |
| 33 | 76.4 |
| 34 | 76.4 |
| 35 | 74 |
| 36 | 74 |
| 37 | 74 |
| 38 | 73.6 |
| 39 | 73.5 |
| 40 | 73.2 |
| 41 | 72.6 |
| 42 | 71.22 |
| 43 | 69.5 |
| 44 | 69.8 |
| 45 | 69.45 |
| 46 | 68.45 |
| 47 | 66.6 |
| 48 | 65.5 |
| 49 | 65.5 |
| 50 | 65.5 |
| 51 | 65.2 |
| 52 | 65 |
| 53 | 65 |
| 54 | 64.5 |
| 55 | 64.8 |
| 56 | 64.8 |
| 57 | 63.8 |
| 58 | 63 |
| 59 | 62.45 |

It is noted that the food extraction apparatuses and methods described above are preferably combined with a behavior modification program that ideally educates patients in modifying caloric intake, lifestyle and attitudes toward food. Learned activities and support for weight loss may include activities such as self-monitoring by recording food intake and physical activity, avoiding triggers that prompt eating, assistance from family and friends, problem solving skills and relapse prevention. The program may be taught by an instructor or offered over the internet. In addition, the program preferably includes a series of regular check-ups by a health care provider. The check-ups ideally include regularly testing blood for electrolytes, supplementing patients' diets with vitamins, and administering medications to prevent gallstone formation as needed. Ideally, the behavior modification program will educate patients to change their lifestyle so as to eliminate the need for food extraction.

The above described embodiments allow obese patients to lose weight without undergoing drastic and invasive surgeries. As a result, obese patients avoid many of the complications associated with such surgeries. In addition, the present invention is easy to perform, easy to reverse and allows obese patients to live a normal and active lifestyle with fewer adverse side effects.

Additional advantages and modifications will readily occur to those skilled in the art. For example, the features of any of the embodiments may be used singularly or in combination with any other of the embodiments of the present invention. In addition, the insertion technique for placing the tube is not limited to known gastrostomy techniques. Moreover, the ePTFE design described herein can also be used for other long-term percutaneous cannula products (e.g. nephrostomy tubes and biliary stents), with application-specific modifications that will be apparent to persons skilled in the relevant arts. Various other modifications may also be made without departing from the spirit or scope of the general inventive concept as defined by the appended claims and their equivalents.

What is claimed is:

1-76. (canceled)

77. A tube comprising:
a. A distal segment adapted to be disposed in an upper digestive system of a patient, the distal segment comprises an elastomeric extruded material and has a distal segment wall thickness; and
b. A proximal segment having a proximal segment wall thickness measuring at least 25% less than the distal segment wall thickness, wherein the proximal segment is configured to pass through the patient's abdominal wall when the distal segment is disposed in the patient's upper digestive system.

78. The tube according to claim 77, wherein the proximal segment comprises a material different from the distal segment.
